(19) United States
(12) Patent Application Publication
(10) Pub. No.: US 2018/0236016 A1
(43) Pub. Date: Aug. 23, 2018

(54) NEUROTRANSMITTER AND BRAIN MODULATING ORAL DELIVERY SYSTEM FOR ENHANCEMENT OF COGNITIVE FUNCTIONS AND ENERGY

(71) Applicant: Steady Nutrition LLC, McLean, VA (US)

(72) Inventor: Aly Gamay, McLean, VA (US)

(73) Assignee: Steady Nutrition LLC, McLean, VA (US)

(21) Appl. No.: 15/896,789

(22) Filed: Feb. 14, 2018

Related U.S. Application Data
(60) Provisional application No. 62/460,760, filed on Feb. 18, 2017.

(51) Int. Cl. A61K 36/185; A61K 31/122; A61K 31/522; A61K 31/185; A61K 31/198; A61K 31/205; A61K 31/365; A61K 36/74; A61K 9/107; A61K 31/20; A61K 47/44; A61K 31/685; A61K 47/26; A61P 25/28; A61K 9/00; A61K 31/197; A61K 31/202; A61K 31/59; A61K 31/355

(52) U.S. Cl. CPC A61K 36/185; A61K 31/355; A61K 31/522; A61K 31/185; A61K 31/198; A61K 31/205; A61K 31/365; A61K 36/74; A61K 9/107; A61K 31/20; A61K 47/44; A61K 31/685; A61K 47/26; A61P 25/28; A61K 9/0053; A61K 31/197; A61K 31/202; A61K 31/59; A61K 31/122

(57) ABSTRACT

Disclosed is a composition and method for making a cognitive function neuromodulation emulsified composition comprising oil soluble and water soluble cognitive function brain modulating source component, a lipid source component, a lipid emulsifier source component a carbohydrate source component, and cocoa source component. Also disclosed does a composition and method for increasing energy and mental focus utilizing a neurotransmitter modulating source component. A method for treating degenerative cognitive functions is also disclosed.

### CROSS-REFERENCE TO RELATED APPLICATIONS

This application claims the benefit of U. S. Provisional Application No. 62/460,760, filed Feb. 18, 2017. The entire disclosure of this prior application is hereby incorporated by reference.

### FIELD OF INVENTION

The present invention relates to compositions, methods of manufacture, and methods of use. Embodiments include oral delivery systems for modulating cognitive brain functions and manipulating neurotransmitters for enhancement of energy, mental health and mood in humans, and methods for treating degenerative cognitive functions.

### BACKGROUND

Current offering of nutraceutical supplements in the market place for the treatment of various ailments and symptoms include: dry pills or capsules (requires long time for dissolving in the stomach, has questionable absorption rate, may require several or large pills), dry powders (require addition to large amount of fluids making them inconvenient to carry or consume, taste is questionable), elixirs and syrups (bulky, hard to carry, unpleasant taste has been a deterrent to broad acceptance by consumers), and chewable tablets and other chews (taste is questionable, smaller dosage than needed may be delivered). Thus, effective delivery systems need to be developed to improve oral methods of delivering therapeutically and nutritionally beneficial compounds.

Poor water solubility is a significant obstacle for nutraceutical supplement absorption. Many nutraceutical supplements are insoluble in water and, therefore, are difficult to formulate. Improving solubility will benefit patients and consumers. Poor water solubility can limit the type of formulation available to a bioactive compound. Poorly soluble nutraceutical supplement may have to be dissolved in oils so that they can be incorporated into a capsule. Poorly soluble compounds are likely to have limited bioavailability. This results in lower absorption and reduced efficacy. To counter this, administration of higher doses is often necessary. However, higher doses can potentially lead to increased side effects.

Time release technology for pills and capsules has been developed to extend the release and provide sustainable supply of compounds. When it comes to liquids like energy drinks and shots, there has not been any timed-release product that has acceptable taste and can keep the functional ingredients in suspension until consumption.

Lipids in food are found in several types of different lipid complexes, with the most abundant one being the triglycerides built from one glycerol molecule and three fatty acids. The lipids in processed foods are normally present as emulsions, which could be utilized to enhance solubility and bioavailability of nutraceuticals.

Human brains are the control center for the bodies and are made up of billions of nerve cells, constantly firing with electrical impulses. Neurotransmitters play a vital part in how brains function. Nerve cells (called neurons) do not actually touch each other. There is a tiny gap between each cell called a synapse. Part of the synapse is called the synaptic cleft. Special chemicals called neurotransmitters relay messages between the neurons. The neuron uses amino acids, vitamins and co-factors to create the chemical/neurotransmitter in the cell, and then passes it to the next neuron. There are many different types of neurotransmitters and each has a specific message. The second neuron accepts the neurotransmitter at a special receptor, much like fitting a key in a lock. Each different neurotransmitter has a different shaped key that fits into a specific lock at the neuron. When it fits into the receptor/lock, it delivers its message. The neuron then produces a neurotransmitter of its own and passes it on to the next nerve cell. Modulating brain function by modulating neurotransmitter production through timed and structured release of amino acids, vitamins, other nutraceuticals and co-factors is a vital developing science to help prevent and treat cognitive diseases and to enhance energy level of individuals.

Neurotransmitters are important for memory, learning, and behavior among other things. There are many types of chemicals that act as neurotransmitters in the human body and the way that foods may affect these chemicals is important to understanding the possible role of diet and supplementation in developmental disorders. Neurotransmitters travel across a synapse to bind to a postsynaptic receptor protein; each neurotransmitter binds only to specific receptors on the postsynaptic membrane. There are different types of receptors for different neurotransmitters. This binding eventually brings about a change in the electrical state of the postsynaptic cell either exciting or inhibiting it. The action can be decreased or neutralized in a number of ways including: glial cells, which remove neurotransmitters from the synaptic cleft; reuptake, where the chemical is taken back to the axon that released it; blocking, whereby the flow by substances that attach to specific receptors is blocked; and by prolonged exposure to the neurotransmitter.

Neurotransmitters include many types such as acetylcholine, which is involved in muscle contractions and in diseases such as myasthenia gravis; serotonin (5-HT), which is synthesized from the amino acid tryptophan which is found in greatest concentration in the gastrointestinal tract and is involved in sensory perception, mood control, depression, impulsivity, aggression, and other behavior problems; dopamine, which is involved in reward or reinforcement, in cognition, and in diseases such as Parkinson's disease, mood disorders, and schizophrenia; norepinephrine, which helps regulate arousal and moods, excites gastrointestinal activity, and modulates endocrine function (e.g., insulin secretion); epinephrine or adrenalin, which is involved in vasoconstriction and dilation, relaxation of smooth muscles of the intestine (thus inhibiting intestinal motility), and endocrine function. The amino acids GABA and glutamate also act as neurotransmitters. GABA is the main inhibitory neurotransmitter reducing anxiety; glutamate is the main excitatory neurotransmitter and is involved in memory formation and in Lou Gehrig's disease. Glutamate is a ubiquitous anionic amino acid that exists in all cell types, but in the brain, it acts as a signaling molecule, being stored and released from the glutamatergic neurons subpopulation. Disturbances in glutamatergic signaling contribute to the pathogenesis of several neurological disorders, such as ischemic stroke, Parkinson's disease, schizophrenia, epilepsy and neurodegenerative disorders. Glutamate is considered the main excitatory neurotransmitter in the central nerve system, used by around half of the neurons in the brain.

In addition to the above neurotransmitters, there are many peptides found in neural tissue that act as neurotransmitters. A number of these are involved in regulating digestion and absorption and are also considered hormones. Some examples of these include: gastrin, which stimulates hydrochloric acid and intestinal motility; CCK, which stimulates pancreatic excretions as well as gallbladder excretion of bile; and secretin, which stimulates release of pancreatic juice and inhibits motility of the intestines.

Aging causes deterioration of various aspects of physiology in normal adults, including memory performance. Impairment of memory performance in the elderly has been detected. Cognitive impairment, progressive decline in cognitive function, changes in brain morphology, and changes in cerebrovascular function are commonly observed in aged or aging individuals. Age-related or age-associated cognitive impairment may manifest itself in many ways, and can include short-term memory loss, diminished capacity to learn or rate of learning, diminished attention, diminished motor performance, and/or dementia. In some cases, a specific etiology of such cognitive decline is unknown, while in other cases, cognitive impairment stems from the onset or progression of recognized diseases, disorders, or syndromes, for example, Alzheimer's disease (AD). Age-associated cognitive decline is distinct from, and can occur independently of AD.

Though there is no universal definition of cognition, it is widely accepted that it may encompass many different components including: concept formation, mental abstraction, language acquisition, text comprehension, higher linguistic abilities, inference, learning, symbolic reasoning, planning, decision making, and metacognition. However, this description may also extend to empathy, procedural memory, introspection, and emotional components relevant to the cognitive process. Globally, cognition relies on a balance d function of several neural circuits and pathways modulated primarily by a range of neurotransmitters.

One recognized syndrome includes age-associated memory impairment due to loss of connectivity between neurons that communicate via neurotransmitters. Enhancing the function of neurotransmitters could be accomplished by enhancing the health of cell membranes and neuron integrity in addition to fueling the neurons with energy. Modern neurology advancement has to focus on modulating both neurons and neurotransmitters.

Millions of Americans suffer from a neurological condition called mild cognitive impairment, wherein lapses in word-finding and name recall are often common, along with other challenges like remembering appointments, difficulty paying bills or losing one's train of thought in the middle of a conversation.

Currently, pharmacological approaches traditionally used to treat medical conditions are used as cognitive enhancers. Neuromodulation methodologies employing invasive and non-invasive procedures can also be used as possible enhancers. Deep brain stimulation is a reversible surgical procedure that implies the insertion of small electrodes in specific brain regions. Biofeedback, or neurofeedback, is one of methodologies used.

As a way to deal with brain fog and tiredness, consumers utilize energy drinks to boost energy and mental focus. The energy drink market is a fast-growing sector of the beverage industry. Individually packaged energy drinks typically contain eight to twenty ounces of liquid drink in a tin can. Another ready to drink form of a concentrated formula is often referred to as an energy shot. Energy shots are popular among consumers who desire energy drinks that are convenient to transport and consume. Energy shots are small enough to easily carry throughout the day and do not require refrigeration, so they are compatible with the increasingly busy lifestyle of the average consumer.

Most energy shots have an unacceptable taste due to very low pH of fluids (about 2.0-3.5), high level of bad tasting actives, lack of masking ability of off flavors due to high water content, and watery mouthfeel. Furthermore, due to the absence of innovative technology for delivering good taste in shelf stable emulsion format, the majority of energy drinks comes only in fruit flavors and offers no option of a rich chocolate taste.

Most of the mental focus and energy drinks on the market rely on stimulants such as caffeine to provide a feeling of increased energy. It would be desirable for those drinks to strengthen other brain functions while they are consumed. Caffeine is a drug that stimulates the central nervous system, heart, muscles, and blood pressure control centers, providing an artificial lift which simulates an increase in energy.

Cocoa and chocolate are highly desirable by consumers who cannot get them in a tasty energy and mental clarity drink. Cocoa and chocolate comprise several advantageous pharmacologically active components, and have been used to alleviate or treat certain disorders. Chocolate consumption has long been associated with enjoyment and pleasure. Popular claims confer on chocolate the properties of being a stimulant, relaxant, euphoriant, aphrodisiac, tonic, and antidepressant. Cocoa may enhance the taste of food and energy preparations if it could be incorporated and remain in a stable form.

This background information is provided for the purpose of providing information believed by the applicant to be of possible relevance to the present invention. No admission is necessarily intended, nor should be construed, that any of the preceding information constitutes prior art against the present invention.

### SUMMARY

Compositions, methods of use, and methods of manufacture are described. Embodiments include compositions and methods of delivering neurotransmitter modulating compounds time-released nutraceutical supplement in an emulsion structure characterized by low water activity. Embodiments further include oral delivery systems for modulating cognitive brain functions and manipulating neurotransmitters to alter energy, mood and mental focus and address degenerative cognitive diseases. Supplements and actives are released in a timely measured pattern. Embodiments include compositions and methods for delivering nutraceutical doses of preserved preparations in low water activity (Aw) and low acidity mediums. Embodiments also include compositions which can be taken orally by a human.

In one embodiment, an edible energy composition comprises theobromine and/or caffeine in an amount from about 30 mg/100 ml to about 700 mg/100 ml, amino acids or an amino acid derivative in an amount from about 20 to about 1200 mg/100 ml, cocoa in an amount from 1000 to about 15000 mg/100 ml, carbohydrate source component in an amount from about 10 to about 50 g100/ml.

In another embodiment, a composition or method described herein is capable of delivering at least one neurotransmitter modulating active to enhance energy, improve memory and/or other cognitive function(s), and/or reduce anxiety in human objects.

In another embodiment, a composition or method described herein is capable of delivering at least one pharmacologically active component in cocoa, and providing mood improvement induced by cocoa or one or more of its pharmacologically active components.

In another embodiment, a composition or method described herein is capable of providing timely release of selected compound(s) into blood stream and enhancing organoleptic characteristics.

In another embodiment, a composition described herein contains a sufficiently low moisture level to keep the active ingredient(s) unexposed to moisture or harmful pH, thus preserving the active ingredient(s) from hydrolysis, dissociation, or other undesirable reaction.

In another embodiment, a composition described herein is capable of dispensing a therapeutically effective dose of active(s) in a drinkable medium that has a low water activity (Aw) of about 0.85 or below to extend its shelf life without adding preservatives and to be stable against temperature fluctuations.

Other objects, features, benefits, and advantages of the present invention will be apparent from the summary and description of embodiments, and will be readily apparent to those skilled in the art having knowledge of gelled products/compositions and their methods of preparation. Such objects, features, benefits, and advantages will be apparent from the above as taken in conjunction with the accompanying examples, tables, data, and all reasonable inferences to be drawn there from.

### Definitions

Unless defined otherwise, all technical and scientific terms used herein have the same meaning as commonly understood by one of ordinary skill in the art to which this invention belongs. Still, certain elements are defined below for the sake of clarity and ease of reference.

“Shelf stable” means a food product that is microbiologically and chemically stable at ambient temperature without refrigeration or freezing.

“Low acid foods” means any food other than alcoholic beverages, with a finished equilibrium pH greater than about 4.6 and a water activity (Aw) greater than about 0.85. Tomatoes and tomato products having a finished equilibrium pH less than 4.6 are not classified as low-acid foods.

The term “pH” is used to designate the intensity or degree of acidity. The value of pH, the logarithm of the reciprocal of the hydrogen ion concentration in solution, is usually determined by measuring the difference potential between two electrodes immersed in a sample solution.

The term “water activity level” is defined in the book, Food Science, by Norman N. Potter, Third Edition, A.V.I. (1984), as a qualitative measure of unbound free water in a system that is available to support biological and chemical reactions. In general, as the water activity of a given food product decreases, its shelf life increases. A high water activity (Aw) product becomes more susceptible to mold, fungus and bacterial proliferation. For instance, the FDA defines a low acid food product with a pH of greater than 4.6 as shelf stable only if it has a water activity of 0.85 or less. Two foods with the same water content can vary significantly in their water activity depending on how much free water is in the system. When a food is in moisture equilibrium with its environment, the water activity of the food will be quantitatively equal to the relative humidity in headspace of the container divided by 100.

The term “viscosity” which is the resistance to flow is mathematically defined as the shear stress divided by the rate of shear strain. Shear stress is the force acting in the plane of the fluid, and shear rate is the velocity gradient of the fluid between the plates. The shear rate takes into account the distance between the plates. It is defined in terms of the force required to move one plane surface continuously past another under specified steady-state conditions when the space between is filled by a specific liquid. Although absolute viscosity can be measured directly if accurate dimensions of the measuring instruments are known, it is more common to calibrate the instrument with a liquid of known viscosity (i.e., standard) and to determine the viscosity of the unknown fluid by comparison with that of the known. Foods exhibit different types of flow. In Newtonian materials, viscosity is not affected by changes in shear rate and remains constant. However, changes in shear rate do affect the viscosity of non-Newtonian materials.

The term “emulsion” means a mixture of two immiscible (unbendable) substances. One substance (the dispersed phase) is dispersed in the other (the continuous phase). Examples of emulsions include butter and mayonnaise. In butter, a continuous liquid phase surrounds droplets of water (water-in-oil emulsion). Emulsification is the process by which emulsions are prepared. Emulsions, being liquids, do not exhibit a static internal structure. The droplets dispersed in the liquid matrix (called the “dispersion medium”) are usually assumed to be statically distributed. Emulsions are part of a more general class of two-phase systems of matter called colloids. Although the terms colloid and emulsion are sometimes used interchangeably, emulsion tends to imply that both the dispersed and the continuous phase are liquid. It is common practice to describe an emulsion as being oil-in-water (O/W) or water-in-oil (W/O).

The term “lipid emulsifiers” means substances that stabilize an emulsion by increasing its kinetic stability and are considered as surfactants or surface active agents. Surfactants are compounds that are amphipathic, having a hydrophilic, water interactive “end,” referred to as their “head group,” and a lipophilic “end,” usually a long chain hydrocarbon fragment, referred to as their “tail.” They congregate at low energy surfaces, including the air-water interface (lowering surface tension) and the surfaces of the water-immiscible droplets found in emulsions (lowering interfacial tension). At these surfaces they naturally orient themselves with their head groups in water and their tails either sticking up and largely out of water (as at the air-water interface) or dissolved in the water-immiscible phase that the water is in contact with (e.g., as the emulsified oil droplet). In both these configurations the head groups strongly interact with water while the tails avoid all contact with water. Surfactant molecules also aggregate in water as micelles with their head groups sticking out and their tails bunched together. Micelles draw oily substances into their hydrophobic cores, explaining the basic action of soaps and detergents used for personal cleanliness and for laundering clothes. The term includes both lipophilic and lipophobic surfactants.

“Timed release,” as used herein, generally refers to release of a drug or nutrient that differs from immediate release of the drug under the same conditions via the same route of administration. Timed release may be modified release, controlled release, extended release, sustained release, or delayed release.

“Sustained release,” as used herein, generally refers to release of a drug whereby the level of drug available to the patient is maintained at some level over a desired period of time.

“HLB” is an empirical expression for the relationship of the hydrophilic and hydrophobic groups of a surface active amphiphilic molecule, such as a surfactant. It is used to index surfactants and its value varies from about 1 to about 20 and includes both non-ionic and ionic surfactants. The higher the HLB value, the more water soluble/dispersible the surfactant is.

“Nutraceuticals” refer to nutritional additives and nutrients including but not limited to vitamins, amino acids, amino peptides, proteins, sports nutritional compounds, phospholipids, minerals, elements, fat-soluble compounds and similar agents for treating conditions previously mentioned.

As used herein, “stability” includes the stability of both the formulation and the therapeutic agent, both prior to administration and after administration to an individual. An improved stability prior to administration enables longer shelf life, less protective packaging, or storage at more aggressive environments.

### DETAILED DESCRIPTION

All conditions characterized as having a need for an ingestible, liquid delivery method can benefit from the compositions and methods of the invention.

The inventive emulsion matrix allows for the single or multiple delivery doses to contain large doses of hydrophilic and nutraceutical preparations. Lipophilicity, fat-liking, refers to the ability of a chemical compound to dissolve in fats, oils, lipids, and non-polar solvents. Thus, lipophilic compounds tend to dissolve lipids. Lipophilicity, hydrophobicity and non-polarity (the latter as used to describe intermolecular interactions and not the separation of charge in dipoles) all essentially describe the same molecular attribute; the terms are often used interchangeably. Hydrophile refers to a physical property of a molecule that can transiently bond with water through hydrogen bonds. A hydrophilic portion of a molecule is one that is typically charge-polarized and capable of hydrogen bonding, enabling it to dissolve more readily in water than in oil or other hydrophobic solvents. Hydrophilic and hydrophobic molecules are also known as polar molecules and non-polar molecules, respectively. Water soluble compounds could be incorporated in the current formulations.

Compounds could be water or fat soluble or not soluble at all, but suspended in the inventive matrix and released in the digestive system. Water soluble ingredients are absorbed quickly into the blood stream almost at the same rate water is absorbed. Depending on the degree of thrust, it could take 5 to 45 minutes for water absorption. Water absorption takes place in the mouth, stomach, small intestine and large intestine. Water soluble compounds will show their impact immediately. Fat soluble compounds will be released after fat digestion has taken place. Fat digestion takes place in a broad range of time ranging from an hour up to seven to thirteen hours to digest depending on the type of emulsion consumed.

As emulsions consists of lipid, water and surfactant, in a specific emulsion, water will harbor water soluble compounds, while fat will harbor fat soluble compounds. Emulsions can be formed with various properties to be digested and released at various intervals. Free water in a food system will be absorbed immediately, bound water (particularly in a low water activity system like the compositions disclosed herein) will be second, while emulsions will release its water later upon digestion, thus releasing a third portion of water. That multiple release pattern will create a gradient of timed release of water soluble compounds for a sustained impact. Lastly, the fourth release will be the fat soluble compounds upon the digestion of lipids in the digestive system at various intervals. A wave of compounds will be released into blood streams creating delayed or timed release. If insoluble actives are used, they will be entrapped within the matrix and delivered to the digestive system where solubilization and absorption takes place. The latter represents the fifth phase of active release.

Emulsions with higher levels of fat will release compounds at a slower rate than emulsions with lower levels of fat, due to prolonged digestion of lipid, which may manipulate the timing of active release.

Another beneficial function of the inventive structured emulsions is their ability to act as carriers of pre-formed fat-soluble vitamins (vitamins A, D, E and K), phospholipids, and other fat soluble or bound compounds.

All emulsions are thermodynamically unstable and may break down into individual phases after certain time periods. Therefore, in practice, the kinetic stability of emulsions is important. Emulsions may become unstable due to a number of different types of physical and chemical processes. In most food emulsions, the droplets are coated by a layer of adsorbed materials, mainly proteins, phospholipids, and low molecular weight emulsifiers, which in turn makes the selection of emulsifiers or surfactants crucial. The emulsion water can cultivate water soluble compounds. The physical properties of adsorbed layers depend on the types, amounts, structures, and interactions of the adsorbed materials. Including proteins from a cocoa or dairy source helps stabilize the emulsion and enhances the strength to hold other low solubility compounds.

One embodiment exemplifies an emulsified composition comprising an oil soluble and water soluble cognitive function brain modulating source component, a lipid source component, a lipid emulsifier source component, a carbohydrate source component, and a cocoa source component without other amino acids incorporation.

Delivery vehicle structure, particularly emulsion properties, can influence the process of lipid digestion and absorption through gastrointestinal tract. Emulsions are exposed to a wide range of conditions when they are first ingested. The taste and the perceived texture of an emulsion depends on the constituent materials used to make the emulsion and on the way the structure behaves in the mouth and how flavors are released, and this in turn necessitates at the same time the development of organoleptically acceptable food matrix for delivery of compounds. In the mouth, the emulsion is mixed with saliva, cools or warms to body temperature, and undergoes changes in pH and ionic strength. The emulsion interacts with various salivary enzymes, experiences the shear effects between the oral mucosa and the teeth, and then passes by peristaltic movement via the esophagus to the gastrointestinal system. Although liquid emulsions stay in the mouth for a very short period of time, they are still exposed to various salivary enzymes, mucilaginous proteins, and salts of various ionic strengths.

By manipulating particle size of fat soluble actives and the main lipid matrix through homogenization and nano homogenization, it is possible to reduce or increase lipid digestion and absorption. Nanoparticle technology could be employed. Nanoemulsions consist of basically miniature sized droplets consisting of a liquid core enveloped in a well-defined membrane. In some embodiments, nanoemulsions have particle sizes between 100 nm to 1,000 nm. Parameters like temperature, pH and electrolyte concentrations could be optimized with respect to different oil:emulsifier ratios (e.g., 10:1, 15:1, 20:1, 25:1 and 30:1), and by adding sodium alginate, a polysaccharide, as encapsulant, and cocoa protein as emulsifier.

Fat emulsions behave differently in the digestive tract depending on their initial physicochemical properties. A lower initial fat droplet size facilitates fat digestion by gastric lipase in the stomach and duodenal lipolysis.

Accordingly, some compositions of the present invention comprise ingredients that provide a modulating effect on solubility, stability, absorption, and metabolism, and may have different PK profiles depending on the drug or nutrient.

The emulsion matrix which is essentially fat soluble compounds and water soluble compounds may entrap water insoluble compounds as well making it deliverable to the stomach for fast ingestion.

In some embodiments, the dietary supplements of the invention provide energy and cognitive benefit by combating the three factors that contribute to a lack of energy: physical fatigue, mental fog, and stress. Physical fatigue can stem from a low rate of metabolism and lack of muscle tone. The slow cellular activity associated with physical fatigue can also hobble the brain and nervous system, causing mental fog. Physical fatigue and mental fog result in poor performances in daily activities and thereby increase stress. Stress in turn burdens the body's systems and exacerbates both physical fatigue and mental fog. Physical fatigue, mental fog, and increased stress levels therefore form a self-perpetuating cycle that drains physical and mental energy. By providing sufficient energy as ketones and various brain stimulating components, the physical, mental and psychological performance are greatly enhanced.

Energy drinks currently on the market may provide a single function such as delivering a high dose of caffeine and other actives that gives a superficial and quick lift to energy that may cause the consumers to feel jittery, anxious, heart palpitation, and unpleasant feeling. Many consumers, especially females, avoid using energy drinks and shots because of palpitations that make a person feel like the heart is beating too hard or too fast, skipping a beat, or fluttering which is bothersome or frightening. In less than a couple of hours, the impact vanishes and a “crash” of energy may occur. The possible explanation of the above is that energy drinks on the market today are all water soluble, where their actives get absorbed into blood stream with absorbed water very quickly (sometimes within minutes) causing a shock to the body. Then the actives get metabolized by the body and lose their impact, and the crash of energy is felt. In some embodiments, the current invention provides a regulated energy active release into blood stream, thus reducing initial shock and maintaining consistent release of energy and mood actives. The impact of the current shot may be felt after about 15 to 30 minutes after consumption and may last for up about to 7 to 9 hours. No jitteriness or crash were reported by test subjects.

The dietary supplements of the present invention may comprise a combination of active ingredients that act synergistically to support the production of neurotransmitters and energize brain cells to achieve optimal brain functioning and improve mental focus. Unlike other energy drinks, which cause mood swings and restlessness, the dietary supplements of the current invention can quiet nervous brain and muscle activity to provide stress relief and sense of well-being.

In some embodiments, the current invention provides delivery of compounds that manipulate neurotransmitters. Many neurotransmitters are considered water-soluble and require transmembrane proteins to transport them across the cell membrane. The endocannabinoids that exist in natural cocoa, like anandamide (AEA) and 2-arachidonoylglycerol (2-AG), are non-charged lipids that readily cross lipid membranes. Since the endocannabinoids are water immiscible, protein transporters act as carriers to solubilize and transport the endocannabinoids through the aqueous cytoplasm. Therefore, the inventive compositions comprising cocoa are capable of delivering both water soluble and fat soluble chemical compounds, such as antidepressant or cognitive function enhancers.

In some embodiments, the composition includes fluid preparations that contain low moisture content (about 5-35%) in a flowable form which allows for dispensing into final package and easy consumption by consumers. Furthermore, the low moisture allows for the incorporation of water sensitive actives without adverse reactions and also masks off-taste and off-flavors associated with bad tasting compounds when ingested. The low temperature of ingredient inclusion and neutral pH of emulsion preserve the nutrient in its original form and state without any physical changes and without conversion to undesirable byproducts. The presence of lipids in the emulsion helps solubilize fat loving components as well as disperse insoluble compounds. Moreover, lipids help calm the digestive system which sometimes experience side effects as a result of consuming large doses of energy nutraceuticals. The low water activity of the delivery matrix ensures long stable shelf life and preservation of actives.

It is possible to mix components of various half-life characteristics in order to ensure a continuous supply of brain supporting components and energy ketones. The sustained release feature of the disclosed emulsions will manipulate the duration of treatment.

In some embodiments, the inventive energy and mental sharpness drinks have a pH of about 5.0 to about 7.5 which is close to neutrality. Other conventional water based energy drinks have a pH of about 2.0 to about 4.0 which is in the acidic range which can cause heartburn and other symptoms associated with consuming acidic beverages. That acidic profile may discourage potential consumers from consuming energy drinks.

In some embodiments, the delivery system of the present invention comprise one or more nutritional and/or energy generating and/or cognitive function brain modulating source component substantially uniformly dispersed within an emulsion matrix which comprises 1) a lipid source component, 2) a carbohydrate source component, 3) a lipid emulsifier source component and 4) cocoa source component. Flavorings, colorings, acidulates, buffers and sweeteners can be included in conventional amounts in the emulsified matrix in order to provide variation in flavor and appearance of emulsions.

In further embodiments, the invention provides a self-dispersing energy and mood nutraceutical composition comprising the following, where the percentages are based on the total weight of the formulation:

1) Surfactant source component: about 0.1% to about 4.5%, or about 1.0% to about 2.5%;

2) Fat source component: about 5% to about 60%, or about 10% to about 25%;

3) Carbohydrate source component: about 1% to about 30%, or about 5% to about 25%;

4) Cocoa source component: about 4% to about 40%, or about 10% to about 25%.

The emulsion matrix of the delivery systems provides minimized degradation of the functional ingredients during the preparation of the matrix and the storage of the final delivery systems as shelf stable. The use of relatively low temperatures in the addition of therapeutic agents ensures that the functional ingredients are not degraded by excessive heat. In accordance with the present invention, the actives may be incorporated at a temperature of 100 F or less. In one embodiment of the present invention, the delivery systems are prepared at or below a temperature of 125 F. In other embodiments, the delivery systems are prepared at or below 170 F.

In addition, the delivery systems maintain a low interaction with water during and after preparation of the matrix, which also contributes to the stability of the functional actives dispersed therein. In accordance with the present invention, the final moisture content of the delivery systems is between about 1% and about 40% by weight. In one embodiment, the final moisture content of the delivery systems is between about 1% and about 10%. In other embodiments, the moisture content is between about 10% and about 15%, between about 15% and about 20% by weight provided the water activity needed to be maintained at a low water activity typically below about 0.85 or under about 0.80.

In one embodiment of the invention, the water activity of the final delivery systems is below about 0.6. In other embodiments, the water activity is below about 0.55, between about 0.45 and about 0.55, between about 0.40 and about 0.55. Low water activity is essential for protecting active agents from degradation at fast pace and provide protection against microbiological spoilage in low acid foods without the exposure to detrimental high heat treatment during processing.

In accordance with the present invention, degradation of the functional ingredients during the process of preparing the matrix is less than about 1%. In one embodiment, degradation of the functional ingredients during preparation of the matrix is less than about 10%. The emulsion matrix also provides for minimized degradation of the functional ingredients dispersed therein during storage of the final delivery systems under normal storage conditions.

The formation of the inventive colloidal emulsion matrix enhances the entrapment of actives and the suspension of water insoluble actives, as well as coats or encapsulates the dry particles and pieces.

The delivery systems of the present invention can be formulated such that the emulsion matrix has a final pH in the range of about 2.5 to about 8.5. In one embodiment, the emulsion matrix has a final pH of between about 5.0 and about 8.5. Acidic pH is known in the art to promote degradation of active ingredients. The inventive delivery system is formulated to deliver agents at neutral to mildly basic. By neutral to mildly basic pH it is meant that the final pH is between about 6.0 and about 8.5. In one embodiment of the present invention, the delivery systems are formulated such that the matrix has a final pH between about 6.2 and about 7.0 and thus are suitable for delivery of acid sensitive actives.

In a final form, the emulsified delivery systems of the present invention could have syrup like consistency or fluid consistency at ambient temperatures depending on the desired application. The present method includes delivery vehicle for energy, mental energy and mood neutraceutical agents coupled with cocoa component in order to maximize and modulate the delivery of the actives within the cocoa and provide building blocks for the stable matrix for treating humans and animals. The composition may be suitable for ingestion by elderly without fear of suffocation. The pleasant taste of the drinkable formulation ensures compliance.

All neurological conditions characterized to have a need for ingestible, drinkable delivery method are able to benefit from the composition and delivery method of the invention. Because of the runny and flowable texture of inventive composition, there is no need to chew a composition; the user just swallows the current composition.

All neurological conditions such as mild cognitive impairment, including lapses in word-finding, name recall and general memory recall may benefit from the compositions and methods described herein. Recall in memory refers to the mental process of retrieval of information from the past. Along with encoding and storage of information, it is one of the three core processes of memory. There are three main types of recall: free recall (the process in which a person is given a list of items to remember and then is tested by being asked to recall them in any order); cued recall (when a person is given a list of items to remember and is then tested with cues to remember material) and Serial recall (the ability to recall items or events in the order in which they occurred. Testing these forms of recall is a way to study the memory processes of humans. The current compositions and methods provide energy as well as essential and complementary compounds to brain cells to improve cognitive function and reduce mild cognitive impairment including recalling.

The current method and compositions are formulated for promoting the neural synthesis release in an animal subject of the neurotransmitters acetylcholine, GABA, glutamate, norepinephrine, dopamine, aspartate, histamine and serotonin. Precursors for each of these neurotransmitters may be administered concomitantly with a xanthine and with one or more precursors for another neurotransmitter selected from precursors for the neurotransmitters histamine, glutamine and aspartate, in order to enhance release of the neurotransmitter in the subject. This procedure for the promotion of synthesis and release of the neurotransmitters may be employed in the treatment of subjects having a neurotransmitter deficiency, including reduced neural tone and excessive neural activity as well cognitive function impairment.

The measurement of viscosity in the inventive emulsified matrix ranges between about between 200 to 10,000 centipoise. In one embodiment, the viscosity was about 100-10,000 centipoise, while in other embodiments measurements of 700 to 3,000 centipoise were obtained.

The nutraceuticals compounds that could be utilized in the method may be pre-hydrated, pre-solubilized, pre-coated, pre-encapsulated, microencapsulated, micronized, particulated, and micro-particulated or prepared as timed-release components either individually or in various combinations.

The disclosed and methods of administering neurotransmitter modulating components can advantageously be used to treat subjects who desire increased energy level, improved mental focus and stamina, sand/or stress relief by actually addressing the underlying causes of fatigue. Positive effect was observed when on or more of the selected nutrients is incorporated in the current delivery system.

### Neurotransmitter Modulating Source Component

The present composition and method can employ numerous types of vitamins, probiotics, hydrolyzed proteins and polypeptides, nutritional supplements synthetic compounds or other nutritional compounds and mixtures thereof in various forms and shapes.

The manufacture and release of these neurotransmitters depends in large part on the concentration of the particular precursor in the blood. Although factors within the brain also control synthesis and function of neurotransmitters, ingestion of particular nutrients can give rise to changes in the neural activity in the brain with resultant changes in physiological functioning and behavior; changes may be subtle in the healthy individual but for those with particular diseases or problems (e.g., depression) they could be significant.

As neurotransmitters can be broadly divided into two main classes: small amino acids (e.g., γ aminobutyric acid (GABA), glutamate, aspartate, tyrosine and glycine) and biogenic amines (e.g., dopamine, epinephrine, norepinephrine, serotonin, histamine, and acetylcholine), all of the above could be incorporated in the current invention.

The current formulations may include cofactors for the synthesis of neurotransmitters such as B vitamins (thiamin, riboflavin, niacin, vitamin B₆, folate, B12), minerals and choline.

Amino acids are precursors for a number of neurotransmitters. Some amino acids, such as tryptophan, are available only from dietary sources. The effect of type of food is most obvious for the amino acids tryptophan, tyrosine, phenylalanine and these amino acids serve as the biosynthetic precursors for the neurotransmitters serotonin, dopamine, and norepinephrine. Amino acids tyrosine and phenylalanine are precursors to dopamine; norephrine is synthesized from dopamine and epinephrine is synthesized from norephrine. Amino acids glutamate and aspartate are neurotransmitters but their level in the brain seems not to be influenced by diet. Choline is the precursor for the neurotransmitter acetylcholine and is found in the form of phosphatidylcholine, or lecithin, in many foods

In another variation of the present embodiment, the energy composition further comprises one or more amino acids or amino acid derivatives. Examples of amino acids or derivatives thereof, include, but are not limited to, N-acetyl L-tyrosine, taurine, L-glutamine, beta alanine, L-carnitine, acetyl L-carnitine, L-theanine, and combinations thereof. Amino acids or derivatives thereof are present in an amount from about 50 mg to about 3500 mg/100 ml. In another refinement, amino acids or derivatives thereof are present in an amount from about 100 mg to about 700 mg/100 ml.

N-Acetyl-L-Cysteine which functions as an antioxidant, is the precursor of glutathione, an even more significant antioxidant and a key detoxifying agent in the liver to reduce the incidence of neurodegenerative disorders such as Alzheimer's and Parkinson's. Dosage: 250 mg to 750 mg/day.

Tyrosine is one of the amino acids, which are the building blocks of protein. The body makes tyrosine from another amino acid phenyl alanine. Tyrosine is used as a supplement to treat an inherited disorder called phenylketonuria, which impair processing phenylalanine properly to make tyrosine needed by the body. Tyrosine is taken for the treatment of depression, attention deficit disorder, attention deficit-hyperactivity disorder, improving alertness following sleep deprivation, Parkinson disease, Alzheimer disease as well as schizophrenia. The body uses tyrosine to make chemical messengers that are involved in conditions involving the brain such as mental alertness. N-acetyl L-tyrosine is a highly bioavailable form of tyrosine due to ease of absorption and transport across the brain barrier. The human brain uses the enzyme tyrosine hydroxylase to convert L-Tyrosine the neurotransmitter dopamine and then to neurotransmitters norepinephrine (noradrenalin) and epinephrine (adrenaline). In an embodiment, tyrosine or N-acetyl L-tyrosine is present in an amount from about 80 mg to about 800 mg/100 ml. Tyrosine or one of its derivatives could be used in the current invention at between about 10 to about 900 mg/100 ml of the drinkable supplement, or at about 150 to about 200 mg per 30 gram serving.

Taurine is the most abundant amino acid in many tissues and, most importantly, in the brain's extracellular fluid. Taurine has been found to impact many different biochemical actions like playing a role in detoxification, membrane stabilization, and osmoregulation, neuropsychiatric illness, treat people with seizure disorders, may provide support for its role in stress, mood, and behavior, use in treating people with seizure disorders, its interaction at GABA receptors demonstrate is sedative effect when used alone. Taurine demonstrates multiple cellular functions including a central role as a neurotransmitter, as a trophic factor in center nerve center development, in maintaining the structural integrity of the membrane, in regulating calcium transport and homeostasis, as an osmolyte, as a neuromodulator and as a neuroprotectant. The neurotransmitter properties of taurine are illustrated by its ability to elicit neuronal hyperpolarization, the presence of specific taurine synthesizing enzyme and receptors in the center nerve center and the presence of a taurine transporter system. Taurine exerts its neuroprotective functions against the glutamate induced excitotoxicity by reducing the glutamate-induced increase of intracellular calcium level when combined with caffeine it may interact in a synergistic way to further increase caffeine's stimulant properties. Taurine is present in an amount from about 100 mg to about 3000 mg/100 ml. Taurine or one of its derivatives could be used in the current invention at between about 500 mg to about 3000 mg/100 ml of the drinkable supplement, or at about 400-800 mg per 30 gram serving.

In another example, the dietary supplement may include L-carnitine or acetyl L-carnitine which plays a vital role in the transport of fats into mitochondria to produce energy. L-carnitine prevents the accumulation of toxins within cells and reduces physical fatigue. L-carnitine is also important in the production of acetylcholine, a neurotransmitter that is a key to memory, intelligence, and mood. The dietary supplements typically include between about 100 to about 1000 mg/100 ml of the drinkable supplement, or at about 400 to about 800 mg of L-carnitine or acetyl L-carnitine per 30 gram serving. Total amino acids incorporated can range from about 20 to about 5000 mg/30 gram serving.

Theobromine is a mild diuretic, a mind stimulant that works on the cardiovascular system, the muscular system and the central nervous system. Theobromine can lower the blood pressure because it can dilute the blood vessels, and also has relaxing effects creating a feeling of wellbeing. Furthermore, theobromine has similar structure to caffeine (both xanthines) but they have differential affinity for adenosine receptor subtypes. In the current invention, both theobromine and caffeine may be mixed to impact long lasting energy feeling. Since caffeine impact is felt in about 15-30 minutes and has a half-life of approximately 5 to 6 hour, while theobromine impact is felt in about 2-3 hours and has a half-life of about 7-12 hours, a combination of the two will provide lasting energy for up to 12 hours. Theobromine has a calming effect which dampens any jitteriness generated by caffeine. Since theobromine is not water soluble it could not be incorporated into conventional water based energy drinks and shots. Moreover, theobromine is soluble in fat require an ideal medium to solubilize this crucial compound.

Caffeine, the most commonly used psychoactive substance to cause the increase in energy, memory and alertness associated with energy drinks. Caffeine is a competitive A1 and A2 adenosine receptor. Adenosine slows down neural activity and, by preventing this inhibitory effect, caffeine stimulates brain activity. The half-life of caffeine (time taken for the body to eliminate one-half of the caffeine) varies widely between people, depending on factors such as age, body weight, pregnancy status, medication intake and liver health. In healthy adults, Caffeine crosses the blood-brain-barrier, which is designed to separate the brain from the bloodstream. Once inside the brain, caffeine blocks the effects of adenosine, which plays an important part in energy transfer and sleep promotion. Caffeine sources include coffee extracts, tea extracts and cocoa.

Caffeine inhibits the activity of the neuroprotective agent adenosine to mask fatigue. The half-life of caffeine (time taken for the body to eliminate one-half of the caffeine) varies widely between people. Caffeine is processed or metabolized in the liver by the cytochrome P450 oxidase enzyme system and broken down into three metabolic dimethylxanthines (paraxanthine, theobromine, and theophylline). These metabolites are then further broken down and excreted in the urine. Caffeine or one of its derivatives could be used in the current invention at between about 5 to about 500 mg/100 ml of the drinkable supplement, or at about 190 mg/100 ml. Theobromine or one of its derivatives could be used in the current invention by itself to produce caffeine free drink or in combination with caffeine. It could be used at between about 10 to about 500 mg/100 ml of the drinkable supplement, or at about 200 mg/100 ml of the drinkable supplement. A combination of theobromine:to caffeine could be accomplished at any ratios ranging between about 1:50 to about 60:1.

The dietary supplements of the invention may comprise GABA, valerian root extract, gingko biloba extract, citrus extract, blueberry leaf extract, turmeric root extract, grape seed extract, periwinkle extract product, wild green oat extract product, and huperzine A extract in amounts such that the supplements are effective for increasing energy, mental focus, reducing stress and supporting the production of neurotransmitters and brain function.

### Cognitive Function Brain Modulating Source Component

A dietary supplement of the invention to enhance cognitive function may further comprise one or more additional ingredients to improve mental focus. Examples of additional ingredients that may be included in the dietary supplements of the invention in order to improve mental focus include, but are not limited to, choline bitartrate and inositol. Choline bitartrate is an essential nutrient that is needed for the manufacture of the acetylcholine. By supporting acetylcholine production, choline bitartrate improves focus and concentration. Inositol aids in the action of the neurotransmitter serotonin, which is known to promote feelings of well-being.

Phospholipids are found in cell membranes as the envelopes which surround and protect the cells in human body. Phosphatidylcholine and phosphatidylserine are an example. Phosphatidylcholine supplies choline to cells, which is needed for the transport of fats in and out of cells. It is also needed for normal brain function, helping to transfer neuro signals from one brain cell to another. The human body can make choline by itself. It is needed to repair damaged cells, for normal brain function, and to transport fat out of fat cells so it can be burned for energy. It is vital for cognitive function, but is not necessarily needed as the body synthesizes it, but at lesser amounts as the body ages. The inventive dietary supplements typically include between about 20 mg to about 550 mg of phosphatidylcholine. Phosphatidylcholine is not water soluble but only lipid soluble. By incorporating into the current emulsion fat, the bioavailability should greatly improve ensuring proper delivery for optimal absorption and transport brain when compared to consuming dry pills or capsule with water. Lecithin which could be used as an emulsifier also provides a significant amount of phosphatidylcholine.

Phosphatidylserine is present in all biological membranes of animals, higher plants and microorganisms. In humans, it is most concentrated in the brain where it comprises 15% of the total phospholipid pool. Phosphatidylserine supplementation in older individuals seems to improve memory and cognitive capacity. Phosphatidylserine is involved in governing membrane fluidity and therefore in the regulation of biological cell activities. As phosphatidylserine exhibits direct and indirect interactions with integral and membrane-associated proteins, it modulates the activity of receptors, enzymes, ion channels and signaling molecules and is vital for neuronal signaling and boosts blood flow to the brain. When the dietary supplements of the invention are administered in a serving size of about one fluid ounce, the dietary supplements typically include between about 20 mg to about 750 mg of PS. PS is not water soluble but only lipid soluble. By incorporating into the current emulsion fat, the bioavailability should greatly improve ensuring proper delivery for optimal absorption and transport brain when compared to consuming dry pills or capsule with water.

In a further example, a dietary supplement may include alpha-glyceryl phosphoryl choline which contains the brain-boosting nutrient choline and can improve memory, thinking, and learning. When the dietary supplements of the invention are administered in a serving size of about one fluid ounce, the dietary supplements typically include between about 20 mg to about 1000 mg of alpha-glyceryl phosphoryl choline.

The dietary supplements may further include fat soluble active like coenzyme Q10 which is a powerful antioxidant that is essential for the production of energy by cells and enhances brain functions. As an antioxidant, it helps neutralize the free radicals that get created during ATP production.

Fat soluble vitamins D and E play a critical role in red blood cell production and maintaining nerve cells as antioxidants.

Omega-3 fatty acids used to manufacture and maintain cell membranes, omega-3s act as anti-inflammatories and mildly thin the blood. Omega-3s come in three major types: Alpha-linolenic acid (ALA), docosahexaenoic acid (DHA), and eicosapentaenoic acid (EPA). Dosage: 200 mg to 2 grams/day.

The current inventive emulsion allows the incorporation of Medium Chain Triglycerides (MCTs), the primary type of fat found within coconut oil, which have been found to boost cognitive performance in older adults suffering from memory disorders as Alzheimer or another type of dementia. Whereas the primary fuel source for the energy-hungry brain is glucose, when insulin resistance and suboptimal metabolism (hypometabolism) develops in the brain, both the brain's structure and function are compromised. Ketone bodies provide a much needed alternative fuel source to glucose that can recharge metabolic processes within the brain, resulting in an almost immediate improvement in cognitive function. The high presence of ketones in the blood stream induces a metabolic state known as ketosis, and in this state cells are able to derive energy not only from glucose (the usual source), but also from the breakdown of ketones. The goal is to have the brain cells tap into that new energy. Due to MCT smaller size they do not form micelles and are not stored in adipose tissue. Whereas up to 97% of the dietary fats we ingest are made up of long-chain triglycerides (LCTs) which have been 14 and 18 carbons, MCTs have relatively shorter chain lengths of 5 to 12 carbons, making them easier to absorb and utilize. They are preferentially oxidized by the liver, and when provided in large enough quantities, they give rise to ketone bodies. Milk fat is considered to be a good source of MCT as well.

The incorporation of MCT in the emulsified composition could possibly improve overall mood state and psychological well-being of individuals.

### Lipid Source Component

Lipid source component used herein means a triglyceride product that provides triglycerides physical characteristics to the beverage concentrates and provide substantially fatty material to whiten the concentrate. The lipids may be of animal, vegetable or mineral origin, which are substantially water-insoluble, inert, non-toxic hydrocarbon fats and oils and derivatives thereof, and may comprise any of the commonly commercially available fats or oils approved by the Food & Drug Administration. The lipid may be a liquid or a solid at room temperature. In some embodiments, the lipid has a melting point in the range of about 90° to about 160° F. (about 32° to about 71° C.). Cocoa butter is an excellent source of lipids as well as chocolates.

The lipid may comprise a vegetable oil base commonly known as hard butter. Hard butters are hydrogenated, press fractionated, or other processed oils that are processed or recombined to have a solid fat index (percent solid fat vs. temperature) similar to that of cocoa butter. However, other lipids may be used that are relatively hard or solid at room temperature, but melt rapidly in the mouth at a temperature of about 92° to 98° F. (29 to 32° C.). The lipid is employed in the amounts within the range of from about 20 to about 50%. Examples of suitable lipids include milk fat, cocoa butter, tallow, hydrogenated tallow, hydrogenated vegetable oil, stearines, almond oil, coconut oil, corn oil, cottonseed oil, fish oil, light liquid petrolatum, heavy liquid petrolatum, olein, olive oil, palm oil, peanut oil, persic oil, sesame oil, soybean oil, castor oil or safflower oil and mixtures thereof. Medium chain fatty acids or enzyme modified fat may also be utilized in the current invention. Lipids maybe added as a part of finished product like chocolate or dairy butter.

One of the pleasant surprises of the current invention discovered is that it is very practical to obtain a ketone source for cognitive activities by adding various Lipid source components like medium chain triglycerides and/or coconut oil.

Depending on the release property desired, a number of natural oils and fats may be used in creating the emulsion at predetermined proportions. For instance, cocoa fat and milk fat might blended to exhibit diversified range of melting point and digestibility and consequently timed release of the actives.

The inclusion of lipid source component in the inventive emulsion enhances the dispersion and delivery of fat soluble or fat immiscible components therefore provide for an improved taste perception of finished emulsion. The fat component works as a masking agent for bitter or astringent nutrients as well as a carrier of added flavors.

The fat component aids in masking off-flavors and unpleasant taste and allows for incorporation of fat soluble flavors and colors for enhancement of organoleptic properties. Furthermore, the fatty slippery structure enhances the flowability during packaging and consumption of finished products.

It was pleasantly surprising to discover that the emulsified lipid source at high levels of about 30 to 50% of finished emulsion composition coats the active particle and prevent further reaction with water or other active ingredients in the matrix at the current low water activity level where most water is bound. The lipid prevents the active water insoluble particles from aggregating and giving hash powdery mouth feel. In other words, the active molecules are segregated by the emulsified lipid component which results in enhanced stability during storage and creamy perception on tongue.

The particular fat source components usage level depends upon a variety of factors such as the desired textural properties in the finished product, total fat level and type, and strength of the matrix. Generally, however, good results are obtained when the total fat source component is present at levels ranging from about 5% to 50%.

### Cocoa Source Component

The term cocoa, used within the context of this invention, includes all such compositions having a significant content of pharmacologically active components present in Theobroma cacao or fermented compositions thereof. Pharmacologically active components from cocoa comprise those components found in cocoa or chocolate and include for example, but not limited to, endogenous cocoa amino acids mixtures, xanthines (e.g. theobromine, caffeine, theophylline), cannabinoid-like fatty acids, biogenic amines (e.g. phenylethylamine, tryptamine, tyramine), epinephrine, norephinephrine, synephrine, minerals (e.g. magnesium) and mixtures thereof.

The cocoa or its pharmacologically active components may be derived from processed or unprocessed cocoa bean (Theobroma cacao). In some embodiments, the cocoa or one or more of its pharmacologically active components comprises or is derived from fermented and subsequently heat-treated cocoa bean. In some embodiments, mixtures of pharmacologically active components isolated from cocoa are used, such as provided in cocoa powder or extract of cocoa. In some embodiments, a cocoa extract is used; such an extract can be obtained by conventional methods using water and/or organic solvents such as hydrocarbons, halogenated hydrocarbons, and alcohols.

Cocoa extracts may be obtained from cocoa beans or cocoa powder by conventional extraction, involving (hot) water extraction, alcohol extraction or extraction using chlorinated hydrocarbons, ketones, esters or other organic solvents. Also, supercritical carbon dioxide may be used as an extracting agent. The extracting agent may be water, alcohol or water/alcohol.

In some embodiments, low fat cocoa product, e.g., cocoa extract having a significant content of pharmacologically active components is used.

Chocolate and cocoa are made up of chemicals associated with mood, cognitive performance, emotion and addiction such as phenylethylamine, theobromine, anandamide and tryptophan trigger mood enhancing chemicals and neurotransmitters to be released in the brain. Phenylethylamine is a chemical found in the body that is similar to amphetamine. It helps mediate feelings of giddiness, attraction, euphoria and excitement. As mentioned previously, theobromine could be provided through the incorporation of cocoa, chocolate or by the addition of natural or synthetic forms.

Furthermore, cocoa comprises cannabinoid-like fatty acids and their derivatives which are chemically and pharmacologically related to anandamide. These cannabinoid-like fatty acids are dissolved in the lipid component of the current invention and are believed subsequently induce mood improvement and/or extend the sense of well-being. In some embodiments, the mood improvement provided by cocoa or one or more of its pharmacologically active components is partially non-serotonin mediated, which subsequently results in a decreased prolactin secretion compared to a situation wherein such mood improvement was solely serotonin mediated. In further embodiments, cocoa powder comprising components capable of increasing serotonin levels and components capable of providing non-serotonin mediated mood improvement. Serotonin (5-HT) increase is followed by an increase in prolactin release and subsequent increase of serum prolactin levels. Cocoa and many of its pharmacologically active components can increase serotonin level compared to a situation where such compositions were not provided. Cocoa for example comprises components capable of inhibiting monoamine oxidase (MAO), components capable of serving as a competitive substrate for MAO and xanthines (e.g. caffeine, theobromine and theophylline). Decreased conversion of serotonin via inhibition of MAO or provision of a competitive MAO substrate will result in elevated serotonin levels compared to a situation wherein such components were not provided. Xanthines (e.g. caffeine) have been shown to increase serotonin levels. Cocoa contains about 20% proteins which will digest in the GI tract to provide additional peptide neurotransmitters.

The naturally occurring theobromine content of cocoa powder (10-12% total fat) is in the range of between 1-2.5% by weight. In plain chocolate there is usually in the order of 0.5-1.5% by weight theobromine. Chocolate also contains tryptophan, an essential amino acid, which plays a role in the production of the neurotransmitter serotonin.

Cocoa may be incorporated at a level of about 1,000 mg to about 20,000 mg/100 ml. Chocolate may be incorporated at levels to provide similar amounts of cocoa per serving. Chocolate liquor may also by incorporate at desired levels.

It was pleasantly discovered that cocoa enhanced the strength of the emulsion at water activity of about 0.85 or lower. Proteins and phospholipids contained in cocoa helped stabilize emulsion. Moreover, organoleptic characteristics of the liquid product were significantly improved by provide mouthfeel, color and bitterness masking properties. Therefore, cocoa is a very essential pharmacologically and functionally component in the drinkable energy and mood enhancing product.

### Carbohydrate Source Component

Suitable materials for use as media are those liquids and fluids with minimal amount of water (about 30% or less) and remain flowable at around 80 to 110 F. Example of such material are sucrose, glucose, fructose, glycerin (about less than 1% water, liquid at room temperature, viscous, stable, hygroscopic, clear, odorless, noncorrosive, and sweet tasting), and polyols (also referred to as sugar alcohols, part of polyols' chemical structure resembles sugar and part is similar to alcohols, the terms polyhydric alcohols and polyalcohols may also be used). Polyols group includes glycerin, maltitol, sorbitol, and xylitol, and mannitol, isomalt and hydrogenated starch hydrolysate. Other examples of suitable media are propylene glycol, lactitol monohydrate, and erythritol. Other suitable media may include syrups of sweeteners such as maltose, fructose, glucose or other mono and disaccharides or natural syrups such as honey, maple syrup and corn syrup.

Carbohydrate source acts as a humectant to reduce the water activity to the recommended level in this method in addition to imparting desirable taste. The level of incorporation could be about 5 to about 60%. In another embodiment, the level was about 10 to about 40%.

Use of a sugar alcohol in place of carbohydrate sweeteners allows for both lowered water activity and lowered sweetness in addition to lowered viscosity. In some embodiments, the sugar alcohol is glycerin. Based on weight, the sugar alcohol may comprise about 5% to about 28% of the weight of finished emulsion-matrix, about to 7% to about 10%, or about 12%.

Sugar alcohols are usually incompletely absorbed into the blood stream from the small intestines which generally results in a smaller change in blood sugar than “regular” sugar sucrose. This property makes them popular sweeteners among diabetics and people on low carbohydrate diets. However, like many other incompletely digestible substances, over consumption of sugar alcohols can lead to bloating and diarrhea because they are not absorbed in the small intestine, however, glycerin is the exception.

Any sweetener could be employed to impart appropriate taste to the finished product. In case a solid emulsion is desired, a gum, polymer or starch ingredient may be added to thicken the texture. Gum Arabic, guar gum, xanthan gum, starch, modified starch, fiber material and/or polymer material may be used individually or in combination.

### Surfactant Source Component

The product of the present invention may be prepared using external emulsifier or surfactant to increase the ease of formation of the emulsion and to promote the stability. Emulsifiers which may be used are those which are approved for use in foods, such as mono- and diglycerides, distilled monoglycerides, lecithin, glycerol mono-stearates, sorbitan esters of hexitol anhydrides, sucrose esters, polyoxyethylene sorbitan esters of hexitol anhydrides, and combinations of such food approved emulsifiers. The amount of emulsifier used varies in the range of about 0.05% to about 3.5%, or about 0.2% to about 1.5% by weight of the emulsion.

The lipid emulsifying agents are different than salt emulsifiers that impact the pH of a product, thus enhancing the buffering capacity resulting in improved stability. There is no need for phosphate or citrate salts or organic chemical emulsifiers to impact textural or acidity modifications. None the less, those agents may be added without harming the current emulsified matrix.

The presence of fat emulsifying agents is essential in order to create a film over the fat globules, which repels any water in the system and allow repulsive forces to keep the lipid suspended and enhance the fat globule membrane or layer intact.

Caseinates are often used as an effective emulsion stabilizer for fats. Moreover calcium caseinate has an HLB value of about 10 and hence it acts as an effective oil-in-water emulsifier. It was pleasantly discovered that protein in cocoa powder functioned similar to caseinate in the inventive emulsified system and the addition of emulsifier may be eliminated. Cocoa powder contains about 20% protein on average. Such protein stabilized the emulsion and provided stability to the structure of the emulsified fats containing active compounds. The advantage of cocoa protein in comparison to caseinate is that caseinate is pH sensitive and may coagulate at pH around or lower than 4.6-4.8 exposing the emulsion to collapsing and losing emulsification properties.

A lipophilic surfactant as defined herein is water insoluble and, in some embodiments, has a hydrophilic-lipophilic balance (HLB) value of less than about 10, less than about 5, or less than about 1 to about 2. Exemplary lipophilic surfactants include, but are not limited to, Maisine 35-1, Imwitor 742, Capmul MCM, and Caproyl 90, a fatty acid selected from the group consisting of octanoic acid, decanoic acid, undecanoic acid, lauric acid, myristic acid, palmitic acid, stearic acid, oleic acid, linoleic acid, and linolenic acid.

A hydrophilic surfactant as defined herein is water soluble and has an HLB value of greater than 10 may be necessary to achieve desirable dispersability of the formulation in the GI tract and release of the therapeutic agent. In this respect, a high HLB surfactant, such as Cremophor RH40, can generally suffice. The levels (amounts) of the high HLB surfactant can be adjusted to provide optimum drug. Exemplary hydrophilic surfactants may include, but are not limited to polyoxyethylene sorbitan fatty acid esters, hydrogenated castor oil ethoxylates, PEG mono- and di-esters of palmitic and stearic acid, fatty acid ethoxylates, and combinations thereof. Formulations of the invention additionally may contain more than one lipophilic surfactant and/or more than one hydrophilic surfactant. In some embodiments, the HLB values of the current invention are about 6 to about 10.

Optional ingredients

Optionally, the present emulsified matrix products can include effective amounts of flavor(s). If present, such flavors can comprise effective amounts of flavors to provide desired flavor levels. Generally, flavors present at from about 0.01% to about 3% of the finished products are contemplated. Suitable non-nutritive sweeteners may also be used for sugar-free fictional foods. Example of non-nutritive sweeteners includes Sucralose, Aspartame, Saccharin and other high potency sweeteners. Suitable materials for use as nutritive carbohydrate sweetening agents are well known in the art. Examples of sweetening agents include both monosaccharide and disaccharide sugars such as sucrose, invert sugar, dextrose, lactose, honey, maltose, fructose, maple syrup and corn syrup or corn syrup solids. Example nutritive carbohydrate sweetening agents include those selected from the group consisting of sucrose, glucose, fructose, and corn syrup solids.

The present compositions can optionally contain a variety of additional ingredients suitable for rendering such products more organoleptically acceptable, more nutritious and/or more storage stable. Such optional components may include colors, coloring agents, preservatives, acidity and pH modifiers (acids and alkaline). Of course, mixtures of the above-noted materials are contemplated herein. Bitter blockers and flavor enhancers may be incorporated to reduce any bitter taste associated with actives.

The present invention may also include glucuronolactone in an amount from about 2 mg to about 1000mg/100 ml. Glucuronolactone is a naturally occurring chemical that is an important structural component of nearly all connective tissues and may be helpful for enhancing cardiovascular functioning, improving athletic performance and promoting alertness.

In some embodiments, the dietary supplements may be administered in liquid form as a drink. A single serving of a dietary supplement of the invention may comprise about one fluid ounce. Advantageously, the dietary supplements in accordance with the invention do not require refrigeration and are shelf stable.

Turning to more of the details, lipid source components could be prepared in advance. If a diversified triglyceride (contains a mixture of naturally produced triglycerols) is desired, selected fat and or/oils are weighted and pre-blended at specified amounts. Lipid source components maybe blended at ambient temperature if it consists mainly of liquid oils or heated up to 150 F if milk fat, cocoa butter or high melting point lipids are incorporated. lipid source components, carbohydrate source components, emulsifier source components, cocoa source components, water and other optional ingredients may be combined in a standard food mixing and processing vessel in any order of addition. Heating is recommended for blending and mixing of various components. The admixture may be heated up to about 150-180 F for about 5-30 minutes and then cooling to about 50-100 F. Any sequence of ingredients addition may be adopted before the incorporation of components. In one embodiment, water is added first to the processor. Next carbohydrate source component and cocoa source components are added with continuous agitation. Next lipid source component and emulsifier source component are added. In some embodiments, the water activity of the mixture is about 0.85 or below, about 0.55 to about 0.85, or about 0.70 to about 0.80.

In some embodiments, the method includes combining the neutraceutical and/or cognitive function brain modulating source component with the admixture at a low temperature of about 50° to about 100° F. or about ambient temperature if the actives are heat sensitive like vitamins. In some embodiments, oil soluble components like Coq10 and fat soluble vitamins like A and E is added at a low temperature below about 120° F., or otherwise the temperature of the system is maintained at about 110° to about 170° F. before the addition of non-heat sensitive components. Amino acids, xanthines, and glucuronolactone may be added at any temperature provided they reach a soluble state.

Optional preparations, flavors, sweeteners, acidity modifiers, colors or other optional ingredients may then be added or at any step of the process. To ensure the active particles are separated and evenly distributed, the ingredients may be blended until a homogenous emulsified configuration is obtained.

In some embodiments, the method comprises a homogenization step. Homogenization helps produce the proper emulsification characteristics and ensure optimal dispersion and distribution of lipid and hydrophobic actives, and the production of a stable product.

While not wishing to be bound by any theory, it is believed that conducting homogenization or nano-emulsification at low water activity of about 0.85 or lower produces surprisingly stable emulsions with appropriate fat droplet size and distribution resulting in sustained release of active(s) in test subjects. For example, when identical energy emulsions of the same actives, but with two different water activities were produced and homogenized, the test subjects reported more sustained energy with the energy product having a lower water activity of 0.83 than the comparable formulation having a water activity of 0.93. It was speculated that the shear forces were much higher in the lower water activity emulsion that delayed breakdown of emulsion and release energy generating actives especially the fat soluble ones at various intervals. It is conceived that the low water activity homogenization or nano-emulsification stabilizes the fat globule layer containing emulsifiers and proteins that causes the emulsion to be digested at different time creating a gradient of active release especially if a mixed type lipid types are utilized.

Single or mixed oil combination could be blended together. After the addition of other components, and adjusting the water activity to about 0.85 or lower, the entire mixture was homogenized at about 1,500 psi (10 MPa) to 60,000 psi (410 MPa) as a single pass or multiple passes for up to 6 hours based on the type of actives and nanoemulsion qualifications required.

In another embodiment, lipid source component and emulsifier source component are added are added first to a mixer at specified ratios. Next cocoa source component is added with continuous agitation. The temperature of the system is maintained at about 70-115° F. before the addition of carbohydrate source component. It is crucial to blend until a homogenous emulsified configuration is obtained. Water activity measurement may be taken at this stage or at the end of the production. Therapeutic agents and nutraceutical actives were added at a temperature lower than 130 F.

The resultant product has a flowable consistency suitable for further filling into suitable containers. A drinkable emulsified chocolate liquid at water activity of about 0.85 may be dispensed at amounts ranging from 10 ml to 100 ml depending on type of fat used and active level needed. A Typical serving is about 20 to 60ml.

Hydrocolloids and starches may be added in suitable amounts to produce a soft consistency. For instance, carboxymethyl cellulose and modified food starch were added to the admixture at about 0.1-1.5% and 2.0-2.5% w/w respectively to produce thicker inventive product.

In some embodiments, the emulsified system has water activity in the range of about 0.50 to about 0.59, and pH of about 5.5 to about 6.5, and/or viscosity in the range of about 2,000 to about 4,000 at about 75° F.

The emulsion-matrix may be filled using any of the filling equipment known to those skilled in the art of packaging technology. Emulsion matrix product may be filled into plastic, glass, and synthetic material, paper or like containers or packages.

The emulsion-matrix may be additionally dispensed into hermetically sealed packages for extended shelf life. Dispensing the compositions into hermetically sealed unit dose offers portability, rigidity, and formability. It also provides protection against moisture, gas and microbiological contamination extending the shelf life of unit dose even after opening the main package. The emulsified preparations may be handled and distributed either at room temperature, refrigerated or frozen depending on the type of active compounds, distribution channels and the end-user.

### Specific Embodiments

The invention encompasses without limitation any one or a combination of the above- and below-indicated aspects and embodiments.

One aspect of the invention provides a composition comprising at least one lipid source component, at least one carbohydrate source component, at least one cocoa source component, at least one oil soluble and water soluble cognitive function brain modulating source component, and at least one lipid emulsifier source component.

Another aspect of the invention provides a composition comprising at least one lipid source component, at least one carbohydrate source component, at least one cocoa source component, and at least one neurotransmitter modulating source component.

In some embodiments, the at least one lipid source component is independently selected from anhydrous milk fat, butter, cream, cocoa butter, partially and fully hydrogenated vegetable fats and oils, cottonseed oil, coconut oil, corn oil, soybean oil, peanut oil, sunflower oil, canola oil, palm kernel oil, tallow, lard, medium chain fatty acids, and enzyme modified fat. In some embodiments, the at least one lipid source component is present at about 1% to about 50%, at least about 5% to about 50%, or at least about 2% to about 40% by weight of the composition.

In some embodiments, the at least one lipid emulsifier source component is independently selected from lecithin mono- and diglycerides, distilled monoglycerides, glycerol mono-stearates, sorbitan esters of hexitol anhydrides, sucrose esters, and polyoxyethylene sorbitan esters of hexitol anhydrides. In some embodiments, the at least one lipid emulsifier is present at about 0.05% to about 1.5% by weight of the composition.

In some embodiments, the at least one carbohydrate source component is independently selected from sucrose, maltose, fructose, glucose, natural syrups, honey, polyols, maltitol, sorbitol, xylitol, mannitol, isomalt, hydrogenated starch hydrolysate, propylene glycol, maltitol monohydrate, erythritol, and glycerin. In some embodiments, the at least one carbohydrate source component is present at about 5% to about 40% by weight of the composition.

In some embodiments, the at least one cocoa source component is independently selected from cocoa powder, chocolate, and chocolate liquor. In some embodiments, the at least one cocoa source component is present in an amount from about 1,000 to about 20,000 mg/100 ml.

In some embodiments, the at least one cognitive function brain modulating source component is independently selected from amino acids, CoQ10, omega-3 fatty acids, medium chain fatty acids, coconut oil, lecithin, cocoa powder, chocolate, chocolate liquor, acetyl L-carnitine, alpha-glyceryl phosphoryl choline, phosphatidylcholine, phosphatidyl serine, N-acetyl-L-cysteine, N acetyl L-tyrosine, and GABA, and is independently pre-hydrated, pre-solubilized, pre-coated, pre-encapsulated, microencapsulated, micronized, particulated, micro-particulated, timed-release, or a combination thereof. In some embodiments, the at least one cognitive function brain modulating source component is present in an amount from about 20 to about 40,000 mg/100 ml.

In some embodiments, the at least one neurotransmitter modulating source component is independently selected from amino acids, caffeine, theobromine, cocoa powder, chocolate, chocolate liquor, glucuronolactone, alpha-glyceryl phosphoryl choline, and phosphatidyl serine, and is independently pre-hydrated, pre-solubilized, pre-coated, pre-encapsulated, microencapsulated, micronized, particulated, micro-particulated, timed-release, or a combination thereof. In some embodiments, the at least one neurotransmitter modulating source component is independently selected from amino acids. In some embodiments, the amino acids are selected from N-acetyl L-tyrosine, taurine, beta alanine, L-carnitine, acetyl L-carnitine, L-theanine, and combinations thereof. In some embodiments, the neurotransmitter modulating source component is present in an amount from about 10 to about 15,000 mg/100 ml, about 20 to about 2000, about 20 to about 1000 mg/100 ml, or about 20 to about 900 mg/100 ml. In some embodiments, the at least one neurotransmitter modulating source component comprises caffeine, and is present in an amount from about 20 to about 1000 mg/100 ml. In some embodiments the at least one neurotransmitter modulating source component comprises theobromine, and is present in an amount from about 20 to about 1000 mg/100 ml. In some embodiments, the at least one neurotransmitter modulating source component comprises glucuronolactone, and is present in an amount from about 10 to about 900 mg/100 ml. In some embodiments, the at least one neurotransmitter modulating source component comprises alpha-glyceryl phosphoryl choline and/or phosphatidyl serine, and is present in an amount from about 20 to about 2000 mg/100 ml.

In some embodiments, the composition further comprises at least one excipient independently selected from sweeteners, buffers, natural and artificial flavorings, and coloring agents.

In some embodiments, the composition further comprises at least one bioactive ingredient independently selected from botanicals, nutritional supplements, vitamins, minerals, hydrolyzed proteins, and polypeptides.

In some embodiments, the composition is liquid at room temperature. In some embodiments, the composition is liquid at ambient temperature. In some embodiments, the composition has a moisture content of between about 0% and about 34%, or about 2% and about 30% by weight of the composition. In some embodiments, the composition has a water activity of less than about 0.85. In some embodiments, the composition has a viscosity of about 1,000 to about 5,000 centipoise, or about 1,000 to about 4,000 centipoise. In some embodiments, the composition has a final pH between about 5.0 and about 8.5.

In some embodiments, the composition is an oil in water emulsified matrix.

Another aspect of the invention provides a method of making an oral delivery composition comprising:

(a) combining at least one neurotransmitter modulating source component, at least one lipid source component, at least one carbohydrate source component, at least one cocoa source component, and water, at a temperature of about 70° to 170° F. to prepare a blend; and

(b) adjusting water activity of the blend to less than about 0.85.

Another aspect of the invention provides a method for treating mild cognitive impairment or degenerative neurological cognitive function comprising administering to a subject in need thereof a composition as described herein.

In some embodiments, the composition is administered at a daily dose of about 30 grams. In some embodiments, the composition is administered for 14 days or longer.

### EXAMPLES

This invention is further illustrated by the following examples, which are to be regarded as illustrative only, and in no way limit the scope of the invention. The following non-limiting examples and data illustrate various aspects and features relating to the method(s) and resulting products/compositions of this invention, including the surprising and unexpected modification, control and/or improvement of the water activity level, homogenization, emulsion formation, solubility of actives, and improvement in energy level and mood elevation of test subjects.

### Example 1

2000 ml of conventional water based energy drink supplements were produced by blending all ingredients below, heating to 170 F and cooling to ambient temperature. About 30 ml of each composition were filled into HDPE plastic bottles. Example 1A contained 210 mg caffeine, 460 mg taurine, 120 mg N Acetyl L-Tyrosine, 300 mg glucuronolactone/30 ml serving. The water activity was 0.95; water content was 86% and pH of 2.90. Sample 1B contained 210 mg theobromine and 0 mg caffeine. Products were filled into 30 ml HDPE bottles and capped. Sample 1B exhibited sedimentation at bottom due to the presence of theobromine which is not water soluble.

| | % | % |
|---|---|---|
| Ingredient | A | B |
| Anhydrous Caffeine | 0.70 | 0.00 |
| Theobromine | 0.00 | 0.70 |
| Taurine | 1.50 | 1.50 |
| N Acetyl L-Tyrosine | 0.40 | 0.40 |
| Glucuronolactone | 1.00 | 1.00 |
| Optional Ingredients | 10.00 | 10.00 |
| Sucralose | 0.30 | 0.30 |
| Water | 86.10 | 86.10 |
| Total | 100.0 | 100.0 |

Bottles of example 1A were given to test subjects who were occasional consumers of energy drinks who were asked to rank taste, feeling of energy, anxiety, mood, and duration of feel of energy, crashing if any. The results indicated that water based energy drink (as in 1A formulation) behaved exactly like conventional water based energy drink in the marketplace. Taste was perceived as being too sour and unpleasant. Some individuals indicated a light headache, jitteriness, feeling of palpitation, anxiety, and fast crashing after about 2 to 3 hours. Mood did not improve, to the contrary impacted by the negative feelings described above. The test subjects indicated that they did not prefer this type of energy generation. Females were more opt to be intolerant to the experience than males.

### Example 2

In order to research the impact of adding energy and mood enhancing ingredients to a dark chocolate base that does not contain added water, 1000 g of the following formulation was evaluated. Chocolate was melted at 120-140 F and actives were stirred in and then the energy chocolate was allowed to cool down to ambient temperature after dividing into 30 g portions.
The products were formulated as follows:

| Ingredient | % |
|---|---|
| Anhydrous Caffeine | 0.70 |
| Theobromine | 0.00 |
| Taurine | 1.50 |
| N Acetyl L-Tyrosine | 0.40 |
| Glucuronolactone | 1.00 |
| Dark Chocolate | 96.40 |
| Total | 100.0 |

The products contained about very low moisture typical of chocolate and had a very low water activity that is negligible.
The organoleptic characteristics of the functional snacks were objectionable and inedible due to the presence of undissolved particles and powders. The above demonstrate that water is essential for the dissolution and solubility of nutraceuticals and an emulsion is an ideal medium to incorporate water soluble ingredients.

### Example 3

Energy stimulating and mood enhancing nutritional supplements were prepared according to the teachings of the current invention.

| | % | % | % |
|---|---|---|---|
| Ingredient | A | B | C |
| Xanthan Gum | 0.00 | 0.05 | 0.10 |
| Mono and Di-Glycerides | 0.30 | 0.00 | 0.50 |
| Anhydrous Milk Fat | 25.00 | 22.00 | 15.00 |
| Cocoa Butter | 2.00 | 0.00 | 10.00 |
| Coffee Extract | 2.00 | 5.00 | 0.00 |
| Anhydrous Caffeine | 0.70 | 0.60 | 1.00 |
| Theobromine | 0.70 | 0.50 | 0.00 |
| Taurine | 2.00 | 2.00 | 1.50 |
| N Acetyl L-Tyrosine | 1.00 | 0.70 | 1.00 |
| Glucuronolactone | 0.50 | 0.60 | 0.60 |
| Cocoa Powder | 8.00 | 7.00 | 15.00 |
| Optional Ingredients | 2.00 | 3.00 | 3.00 |
| Sugar | 30.00 | 29.00 | 20.00 |
| Glycerine | 2.00 | 5.00 | 10.00 |
| Water | 23.80 | 24.15 | 22.30 |
| Total | 100.0 | 100.0 | 100.0 |

For each formulation, 1000 g batches were made. Milk fat and cocoa butter were weighted up and pre-blended at the specified amounts, then heated up to 130 F in a processing vessel. Water, mono and diglycerides, sugar, glycerin, cocoa powder and other optional ingredients were added Caffeine, theobromine, glucuronolactone, taurine and N acetyl L-tyrosine were mixed in. The admixture was heated up to 170-180 F for about 5 minutes and then cooled to 100 F. The water activity was adjusted to 0.85 or lower by the addition of glycerin. The entire admixture was homogenized at 1,000 psi for example 3A and at to 3,000 psi for example 3B and 3C. The products were filled into 30 ml HDPE Boston round bottles. Water activity readings were 0.81, 0.82 and 0.72 for example 3A, 3B and 3C respectively. The pH readings were 5.8, 5.3 and 6.7 for example 3A, 3B and 3C respectively. Viscosity readings were 110, 170 and 250 centipoise for example 3A, 3B and 3C respectively. Theobromine was not precipitated as it was mostly solubilized in the fat phase and suspended in the viscous emulsion structure.

Bottles of example 3A were chosen to be given to test subjects who were occasional consumers of energy drinks who were asked to rank taste, the feeling of energy, anxiety, mood, and duration of feel of energy, crashing if any. The results indicated that the inventive energy drink behaved quite differently than conventional water based energy drink. Taste was extremely liked and was perceived as liquid chocolate syrup with a hint of coffee. Some individuals indicated the tendency to consume an energy drink that taste sweet, chocolaty, creamy and indulging.

There were no reports of any headache, jitteriness, feeling of palpitation, anxiety. The energy onset took longer than water based energy drinks and started to kick in after about 15 to 30 minutes and continued to build up as a sustained energy pattern. Mood enhancement was reported; many individuals reported a “feeling good” attitude. The mood and energy impact lasted for about 5 to 8 hours as reported. Test subjects indicated a “soft landing” at the end instead of “crashing” No loss of sleepiness or insomnia was conveyed. The test subjects indicated that they prefer this type of timed release of energy. Females were more impressed than males by the new discovery of indulging and tolerable energy drink that is portable and convenient. Moreover, any male individuals reported their satisfaction with the type of energy and mood the inventive product delivered. Mental focus and sharpness were also mentioned as additional advantages of the current inventive product.

### Example 4

Decaffeinated energy and cognitive focus drinks were formulated as shown below. This example further illustrates the potential of including various fat and water soluble components to attain more beneficial health attributes. Same procedures described above were followed to produce the products.

| | % | % | % |
|---|---|---|---|
| Ingredient | A | B | C |
| Polysorbate 60 | 0.10 | 0.20 | 0.20 |
| Medium Chain Fatty Acids | 5.00 | 2.00 | 0.00 |
| Anhydrous Milk Fat | 15.00 | 25.00 | 45.00 |
| Theobromine | 0.70 | 1.00 | 1.00 |
| Taurine | 2.00 | 2.50 | 1.50 |
| N Acetyl L-Tyrosine | 1.00 | 1.50 | 0.30 |
| Glucuronolactone | 0.50 | 1.00 | 0.30 |
| Coenzyme Q10 | 0.00 | 0.00 | 1.20 |
| Cocoa Powder | 8.00 | 7.00 | 6.00 |
| L-Carnitine | 0.00 | 1.10 | 0.00 |
| Optional Ingredients | 2.00 | 2.00 | 5.00 |
| Sugar | 30.00 | 25.00 | 20.00 |
| Glycerin | 3.00 | 8.00 | 7.00 |
| Water | 32.70 | 23.70 | 12.50 |
| Total | 100.0 | 100.0 | 100.0 |

The inventive resultant products did enhance mental energy, physical daily performance as well as stamina and mental focus as reported by test subjects. Perception of memory improvement was also mentioned by a few participants.

### Example 5

Neuroprotective and cognitive nutritional supplements were prepared according to the teachings of the current invention:

| | % | % | % |
|---|---|---|---|
| Ingredient | A | B | C |
| Lecithin | 0.50 | 0.00 | 1.00 |
| Medium Chain Fatty Acids | 15.00 | 20.00 | 10.00 |
| Coconut Oil | 10.00 | 10.00 | 15.00 |
| Cocoa Powder | 8.00 | 8.00 | 6.00 |
| Acetyl L-Carnitine | 1.20 | 0.00 | 0.00 |
| Phosphatidylcholine | 1.50 | 1.00 | 1.00 |
| PhosphatidylSerine | 2.10 | 2.50 | 2.00 |
| N Acetyl L-Tyrosine | 0.00 | 1.00 | 1.20 |
| Alpha glyceryl Phosphoryl Choline | 1.80 | 2.50 | 1.80 |
| Eicosapentaenoic Acid | 0.00 | 2.20 | 2.20 |
| Vitamin D and E Blend | 0.01 | 0.01 | 0.01 |
| N-Acetyl-L-Cysteine | 0.00 | 1.20 | 0.00 |
| GABA | 0.00 | 0.00 | 0.50 |
| Glutamine | 0.00 | 1.00 | 0.50 |
| Optional Ingredients | 7.00 | 7.00 | 7.00 |
| Glycerine | 17.00 | 15.00 | 22.00 |
| Water | 35.89 | 27.99 | 29.79 |
| Total | 100.0 | 100.0 | 100.0 |

For each formulation, 1000 g batches were made. MCT and coconut oil were weighted up and pre-blended at the specified amounts, then heated up to 130 F in a processing vessel. Lecithin, oil soluble actives like eicosapentaenoic acid, phosphatidylserine and phosphatidylcholine were mixed in. Water, glycerin, cocoa powder and other optional ingredients were added. Water soluble cognitive function brain modulating source component was mixed in. The admixture was heated up to 170-180 F for about 5 minutes and then cooled to 100 F. Vitamin D and E were then added. The water activity was adjusted to 0.85 or lower by the addition of glycerin. The entire admixture was homogenized at 30,000 psi to produce fat droplet with sizes between 100 nm to 1,000 nm. The products were filled into 30 ml HDPE Boston round bottles. Water activity reading was 0.84 to 0.79. The pH reading was 5.2 to 6. Viscosity reading ranged between 190 and 450 centipoise. The drinkable liquid was packaged in 30 ml vials.

### Example 6

A method for treating impairment of neurological degenerative cognitive functions is described. Mild cognitive impairment, including lapses in word-finding, name recall and general memory recall may benefited from the method described herein.

Bottles of example 5A were chosen to be given to test subjects who were asked to provide feedback on feeling of reduction of anxiety, enhancement of well-being, and recall in memory function after ingesting one 30 ml serving for fourteen days. The results indicated that the inventive method improved well-being perception and recall in memory function. Lower perception of anxiety was also reported.

What is claimed is:

1. An emulsified composition comprising at least one oil soluble and water soluble cognitive function brain modulating source component, at least one lipid source component, at least one lipid emulsifier source component, at least one carbohydrate source component, and at least one cocoa source component.

2. The composition of claim 1, wherein the at least one cognitive function brain modulating source component is independently selected from amino acids, CoQ 10, omega-3 fatty acids, medium chain fatty acids, coconut oil, lecithin, cocoa powder, chocolate, chocolate liquor, acetyl L-carnitine, alpha-glyceryl phosphoryl choline, phosphatidylcholine, phosphatidyl serine, N-acetyl-L-cysteine, N acetyl L-tyrosine, and GABA, and is independently pre-hydrated, pre-solubilized, pre-coated, pre-encapsulated, microencapsulated, micronized, particulated, micro-particulated, timed-release, or a combination thereof.

3. The composition of claim 1, wherein the at least one lipid source component is independently selected from anhydrous milk fat, butter, cream, cocoa butter, partially and fully hydrogenated vegetable fats and oils, cottonseed oil, coconut oil, corn oil, soybean oil, peanut oil, sunflower oil, canola oil, palm kernel oil, tallow, lard, medium chain fatty acids, and enzyme modified fat, and is present at about 5% to about 50% by weight of the composition.

4. The composition of claim 1, wherein the at least one lipid emulsifier source component is independently selected from lecithin mono- and diglycerides, distilled monoglycerides, glycerol mono-stearates, sorbitan esters of hexitol anhydrides, sucrose esters, and polyoxyethylene sorbitan esters of hexitol anhydrides, and is present at about 0.05% to about 1.5% by weight of the composition.

5. The composition of claim 1, wherein the at least one carbohydrate source component is independently selected from sucrose, maltose, fructose, glucose, natural syrups, honey, polyols, maltitol, sorbitol, xylitol, mannitol, isomalt, hydrogenated starch hydrolysate, propylene glycol, maltitol monohydrate, erythritol, and glycerin and is present at about 5% to about 40% by weight of the composition.

6. The composition of claim 1, wherein the at least one cocoa source component is independently selected from cocoa powder, chocolate, and chocolate liquor.

7. The composition of claim 1, further comprising at least one excipient independently selected from sweeteners, buffers, natural and artificial flavorings, and coloring agents.

8. The composition of claim 1, further comprising at least one bioactive ingredient independently selected from botanicals, nutritional supplements, vitamins, minerals, hydrolyzed proteins, and polypeptides.

9. The composition of claim 2, wherein the at least one cognitive function brain modulating source component is present in an amount from about 20 to about 40,000 mg/100 ml.

10. The composition of claim 1, wherein the at least one cocoa source component is present in an amount from about 1,000 to about 20,000 mg/100 ml.

11. The composition of claim 1, wherein the composition is liquid at room temperature.

12. The composition of claim 1, wherein the composition has a moisture content of between about 2% and about 30% by weight.

13. The composition of claim 1, wherein the composition has a water activity of less than about 0.85.

14. The composition of claim 1, wherein the composition has a viscosity of about 1,000 to about 4,000 centipoise.

15. The composition of claim 1, wherein the composition has a final pH between about 5.0 and about 8.5.

16. A composition comprising at least one neurotransmitter modulating source component, at least one lipid source component, at least one carbohydrate source component, and at least one cocoa source component.

17. The composition of claim 16, wherein the composition is an oil in water emulsified matrix.

18. The composition of claim 16, wherein the at least one neurotransmitter modulating source component is independently selected from amino acids, caffeine, theobromine, cocoa powder, chocolate, chocolate liquor, glucuronolactone, alpha-glyceryl phosphoryl choline, and phosphatidyl serine, and is independently pre-hydrated, pre-solubilized, pre-coated, pre-encapsulated, microencapsulated, micronized, particulated, micro-particulated, timed-release, or a combination thereof.

19. The composition of claim 18, wherein the amino acids are selected from N-acetyl L-tyrosine, taurine, beta alanine, L-carnitine, acetyl L-carnitine, L-theanine, and combinations thereof.

20. The composition of claim 16, wherein the one or more lipid source component is independently selected from anhydrous milk fat, butter, cream, cocoa butter, partially and fully hydrogenated vegetable fats and oils, cottonseed oil, coconut oil, corn oil, soybean oil, peanut oil, sunflower oil, canola oil, palm kernel oil, tallow, lard, medium chain fatty acids, and enzyme modified fat, and is present at about 2% to about 40% by weight of the composition.

21. The composition of claim 16, wherein the at least one carbohydrate source component is independently selected from sucrose, maltose, fructose, glucose, natural syrups, honey, polyols, maltitol, sorbitol, xylitol, mannitol, isomalt, hydrogenated starch hydrolysate, propylene glycol, maltitol monohydrate, erythritol, and glycerin, and is present at about 5% to about 40% by weight of the composition.

22. The composition of claim 16, wherein the at least one cocoa source component is independently selected from cocoa powder, chocolate, and chocolate liquor.

23. The composition of claim 16, further comprising at least one excipient independently selected from sweeteners, buffers, natural and artificial flavorings, and coloring agents.

24. The composition of claim 16, further comprising at least one bioactive ingredient independently selected from botanicals, nutritional supplements, vitamins, minerals, proteins, and polypeptides.

25. The composition of claim 19, wherein the at least one neurotransmitter modulating source component is independently selected from said amino acids, and is present in an amount from about 20 to about 15,000 mg/100 ml.

26. The composition of claim 18, wherein the at least one neurotransmitter modulating source component comprises said caffeine, and is present in an amount from about 20 to about 1000 mg/100 ml.

27. The composition of claim 18, wherein the at least one neurotransmitter modulating source component comprises said theobromine, and is present in an amount from about 20 to about 1000 mg/100 ml.

28. The composition of claim 18, wherein the at least one neurotransmitter modulating source component comprises said glucuronolactone, and is present in an amount from about 10 to about 900 mg/100 ml.

29. The composition of claim 18, wherein the at least one neurotransmitter modulating source component comprises said alpha-glyceryl phosphoryl choline and/or said phosphatidyl serine, and is present in an amount from about 20 to about 2000 mg/100 ml.

30. The composition of claim 16, wherein the composition is liquid at ambient temperature.

31. The composition of claim 16, wherein the composition has a moisture content of between about 0% and about 34% by weight.

32. The composition of claim 16, wherein the composition has a water activity of less than about 0.85.

33. The composition of claim 16, wherein the composition has a viscosity of about 1,000 to 5,500 centipoise.

34. The composition of claim 16, wherein the composition has a final pH between about 5.0 and about 8.5.

35. A method of making an oral delivery composition comprising:
(a) combining at least one neurotransmitter modulating source component, at least one lipid source component, at least one carbohydrate source component, at least one cocoa source component, and water, at a temperature of about 70° to 170° F. to prepare a blend; and
(b) adjusting water activity of the blend to less than about 0.85.

36. The method of claim 35, wherein the at least one neurotransmitter modulating source component is independently selected from amino acids, caffeine, theobromine, cocoa powder, chocolate, chocolate liquor, glucuronolactone, alpha-glyceryl phosphoryl choline, and phosphatidyl serine, and is independently pre-hydrated, pre-solubilized, pre-coated, pre-encapsulated, microencapsulated, micronized, particulated, micro-particulated, timed-release, or a combination thereof.

37. The composition of claim 36, wherein the amino acids are selected from N-acetyl L-tyrosine, taurine, beta alanine, L-carnitine, acetyl L-carnitine, L-theanine, and combinations thereof.

38. The composition of claim 35, wherein the at least one lipid source component is independently selected from anhydrous milk fat, butter, cream, cocoa butter, partially and fully hydrogenated vegetable fats and oils, cottonseed oil, coconut oil, corn oil, soybean oil, peanut oil, sunflower oil, canola oil, palm kernel oil, tallow, lard, medium chain fatty acids, and enzyme modified fat, and is present at about 2% to about 40% by weight of the composition.

39. The composition of claim 35, wherein the at least one carbohydrate source component is independently selected from sucrose, maltose, fructose, glucose, natural syrups, honey, polyols, maltitol, sorbitol, xylitol, mannitol, isomalt, hydrogenated starch hydrolysate, propylene glycol, maltitol monohydrate, erythritol, and glycerin, and is present at about 5% to about 40% by weight of the composition.

40. The composition of claim 35, wherein the at least one cocoa source component is independently selected from cocoa powder, chocolate, and chocolate liquor.

41. The composition of claim 35, further comprising at least one excipient independently selected from sweeteners, buffers, natural and artificial flavorings, and coloring agents.

42. The composition of claim 36, further comprising at least one bioactive ingredient independently selected from botanicals, nutritional supplements, vitamins, minerals, proteins, and polypeptides.

43. The composition of claim 37, wherein the at least one neurotransmitter modulating source component is independently selected from said amino acids, and is present in an amount from about 20 to about 15,000 mg/100 ml.

44. The composition of claim 36, wherein the at least one neurotransmitter modulating source component comprises said caffeine, and is present in an amount from about 20 to about 1000 mg/100 ml.

45. The composition of claim 36, wherein the at least one neurotransmitter modulating source component comprises said theobromine, and is present in an amount from about 20 to about 1000 mg/100 ml.

46. The composition of claim 36, wherein the at least one neurotransmitter modulating source component comprises said glucuronolactone, and is present in an amount from about 10 to about 900 mg/100 ml.

47. The composition of claim 36, wherein the at least one neurotransmitter modulating source component comprises said alpha-glyceryl phosphoryl choline and/or said phosphatidyl serine, and is present in an amount from about 20 to about 2000 mg/100 ml.

48. The composition of claim 35, wherein the composition is liquid at ambient temperature.

49. The composition of claim 35, wherein the composition has a viscosity of about 1,000 to about 5,500 centipoise.

50. The composition of claim 35, wherein the composition has a final pH between about 5.0 and about 8.5.

51. A method for treating mild cognitive impairment or degenerative neurological cognitive function comprising administering to a subject in need thereof the composition of claim 1 at a daily dose of about 30 grams.

52. The method of claim 51, wherein the composition is administered for 14 days or longer.

53. A method for treating mild cognitive impairment or degenerative neurological cognitive function comprising administering to a subject in need thereof the composition of claim 16 at a daily dose of about 30 grams.

54. The method of claim 53, wherein the composition is administered for 14 days or longer.
